(19) United States
(12) Patent Application Publication
(10) Pub. No.: US 2026/0364247 A1
(43) Pub. Date: Nov. 19, 2026

(54) RAPID INFUSION WORKFLOW PROMOTION

(71) Applicant: CareFusion 303, Inc., San Diego, CA (US)

(72) Inventors: Michael K. WORKMAN, Carlsbad, CA (US); Jeffrey GAETANO, San Diego, CA (US)

(21) Appl. No.: 19/210,697

(22) Filed: May 16, 2025

(51) Int. Cl. A61M 5/142; A61M 5/172

(52) U.S. Cl. CPC A61M 5/142; A61M 5/172; A61M 2005/14208; A61M 2205/505; A61M 2205/52

(57) ABSTRACT

An infusion device is started in a first mode whereby the type of infusion is unknown to the infusion device. A trigger condition is generated based on a technical factor associated with the infusion device or a clinical factor associated with a patient receiving the infusion or a care area or clinician associated with the infusion device and, responsive to the trigger condition being satisfied, a graphical window is displayed that presents a control configured to, when activated, reduce a predetermined number of workflow steps that are required to identify the type of the infusion and to switch the infusion device from the first mode to a second mode. The control is activated to enter the type of the infusion via the reduced number of workflow steps and the infusion device is switched from the first mode to the second mode based on the type of the infusion.

### BACKGROUND

This application relates generally to ensuring that an infusion device is properly programmed.

Modern day infusion devices may operate in multiple modes. For example, an infusion device may operate in a smart mode, which takes advantage of drug libraries and smart guiderails (e.g., limits) as well as patient-specific calculations, or may operate in a basic mode in which nothing is known about the patient or the drug being infused. The basic mode allows the infusion pump to be easily and quickly set up in emergency situations (e.g., by merely setting a flow rate and/or volume to be infused (VTBI)). However, once programmed for a basic infusion, the infusion device may be left to run in this mode all day, and it is common for clinicians to forget to promote the infusion device to the smart mode, thus leaving the infusion device in this mode for extended periods of time. Moreover, updating the pump to function in the smart mode and/or to use the correct parameters generally requires an extended series of workflow steps that tend to discourage clinicians from performing the update.

### SUMMARY

The subject technology provides a system and method for facilitating a rapid promotion of an ongoing infusion from a first mode to a second mode. One problem discouraging clinicians from promoting an ongoing infusion (e.g., by updating its parameters) from a first mode to a second, more advanced mode includes the excess number of workflow steps required to perform the update. This problem is compounded by the lack of any particular alert mechanism for ensuring that the pump will be updated with the correct parameters to switch to the second mode.

According to various implementations, the subject technology provides an infusion device configured to: detect an infusion was initiated by the infusion device in a first mode, wherein when the infusion device is in the first mode a type of the infusion is unknown to the infusion device; cause, on a display device associated with the infusion device, a display of a user interface comprising real time information pertaining to the infusion, the user interface providing an interactive workflow comprising a number of workflow steps that are required to identify the type of the infusion and to switch the infusion device from the first mode to a second mode that is based on the identified type of the infusion; when the infusion is initiated in the first mode: identify at least one technical factor associated with the infusion device or at least one clinical factor associated with a patient receiving the infusion or a care area or clinician associated with the infusion device; and generate a trigger condition based on the identified at least one technical factor or the at least one clinical factor; responsive to the trigger condition being satisfied, automatically cause, on the display device, a display of a graphical window that presents a control configured to, when activated, reduce the number of workflow steps that are required to identify the type of the infusion and to switch the infusion device from the first mode to a second mode; receive, after the graphical window is displayed, an indication that the control was activated and that the type of the infusion was identified via the reduced number of workflow steps being completed by a user; and switch the infusion device from the first mode to the second mode based on the type of the infusion and the reduced number of workflow steps being completed by the user. Other aspects include corresponding methods, systems and computer program products for implementation of the corresponding device and its features.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description of Implementations below, in conjunction with the following drawings. Like reference numerals refer to corresponding parts throughout the figures and description.

FIG. 1A depicts an example of an institutional patient care system of a healthcare organization, according to aspects of the subject technology.

FIG. 1B is another view of a portion of the example patient care unit shown in FIG. 1A, according to aspects of the subject technology.

FIG. 2 depicts an example sequence diagram of providing an infusion promotion reminder for rapid infusion workflow promotion, according to various aspects of the subject technology.

FIG. 3 depicts an example flow diagram for a process of providing an infusion promotion reminder for rapid infusion workflow promotion, according to aspects of the subject technology.

FIG. 4 depicts an example flow diagram for a process of rapid infusion workflow promotion, according to aspects of the subject technology.

FIG. 5 is a conceptual diagram illustrating an example electronic system for rapid infusion workflow promotion, according to aspects of the subject technology.

### DESCRIPTION

Reference will now be made to implementations, examples of which are illustrated in the accompanying drawings. In the following description, numerous specific details are set forth in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

Changing the operating mode of an infusion device generally involves multiple workflow steps which clinicians often avoid due to time constraints and other human factors. The subject technology provides a mechanism to remind clinicians that a mode should be changed—that is, promoted—and an expedited workflow process for effectuating the promotion. In this regard, the subject technology sets an internal promotion trigger condition when an infusion is first initiated in a first, basic mode that does not require entry of certain parameters (e.g., patient identity and/or medication type). When the trigger condition is satisfied (e.g., after a predetermined time), the infusion device will present a promotion reminder on a display associated with the device, and provide a control for bypassing workflow steps to promote the mode of the device.

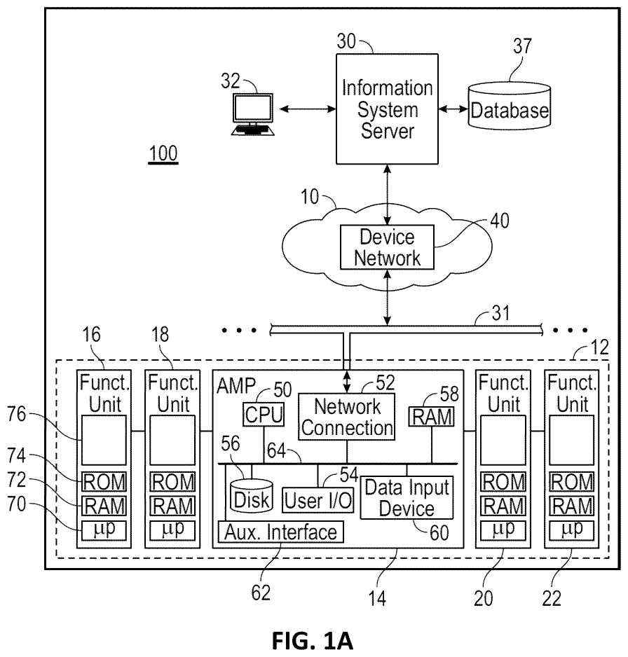

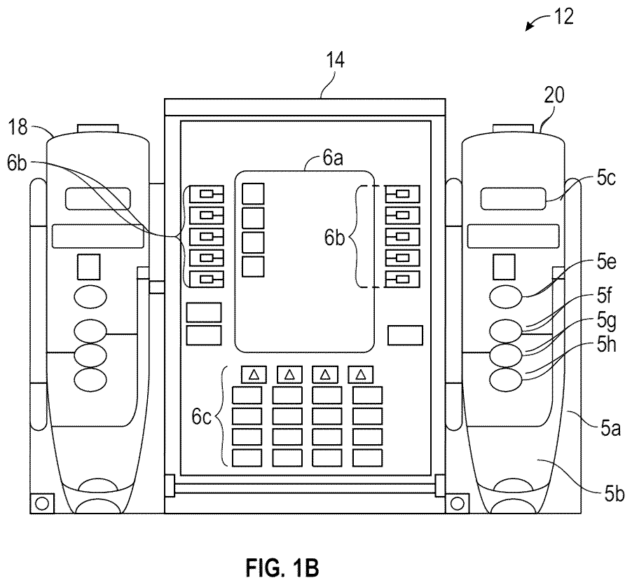

FIG. 1A depicts an example of an institutional patient care system 100 of a healthcare organization, according to aspects of the subject technology. In FIG. 1A, a patient care unit (“PCU” or “medical device” generally) 12 is connected to a hospital network 10. The term patient care unit may include various ancillary medical devices such as an infusion device (including a pump), vital signs monitor, a medication dispensing device (e.g., cabinet, tote), a medication preparation device, an automated dispensing device, a module coupled with one of the aforementioned (e.g., a syringe pump module configured to attach to an infusion pump), or other similar devices. The PCU 12 may be connected to an internal healthcare network 10 by a network transmission channel 31. Network transmission channel 31 is any wired or wireless transmission channel, for example an 802.11 standard-based wireless local area network (LAN). In some implementations, network 10 also includes computer systems located in various departments throughout a hospital. For example, network 10 of FIG. 1A optionally includes computer systems associated with an admissions department, a billing department, a biomedical engineering department, a clinical laboratory, a central supply department, one or more unit station computers and/or a medical decision support system. As described further below, network 10 may include discrete subnetworks. In the depicted example, network 10 includes a device network 41 by which patient care units 12 (and other devices) communicate in accordance with normal operations.

Additionally, institutional patient care system 100 may incorporate a separate information system server 30. Moreover, although the information system server 30 is shown as a separate server, the functions and programming of the information system server 30 may be incorporated into another computer, if such is desired by engineers designing the institution's information system. Institutional patient care system 100 may further include one or multiple device terminals 32 for connecting and communicating with information system server 30. Device terminals 32 may include personal computers, personal data assistances, and mobile devices such as laptops, tablet computers, augmented reality devices, or smartphones, configured with software for communications with information system server 30 via network 10.

Patient care unit 12 comprises a system for providing patient care. Patient care unit 12 may include or incorporate operating system components such as pumps, physiological monitors (e.g., heart rate, blood pressure, ECG, EEG, pulse oximeter, and other patient monitors), therapy devices, and other drug delivery devices may be utilized according to the teachings set forth herein. In the depicted example, patient care unit 12 comprises a control module 14 connected to one or more functional modules 16, 18, 20, 22. Control unit 14 includes one or more operating systems including, for example, a central processing unit (CPU) 50 connected to a memory, such as a random access memory (RAM) 58, and one or more interface devices such as a user interface device 54, a coded data input device 60, and an auxiliary interface 62 for communicating with additional modules or devices. Control unit 14 (and/or its operational systems) may also include a main non-volatile storage unit 56, such as a hard disk drive or non-volatile flash memory, for storing software and data and one or more internal buses 64 for interconnecting the aforementioned elements.

In various implementations, user interface device 54 is a touch screen for displaying information to a user and allowing a user to input information by touching defined areas of the screen (e.g., 6a of FIG. 1B). Additionally, or in the alternative, user interface device 54 could include any means for displaying and inputting information, such as a monitor, a printer, a keyboard, softkeys, a mouse, a track ball and/or a light pen. Data input device 60 may be a code reader capable of scanning and interpreting data printed in a coded format (e.g., a scancode). Additionally or in the alternative, data input device 60 can be any device for entering coded data into a computer, such as a device(s) for reading a magnetic strips, radio-frequency identification (RFID) devices whereby digital data encoded in RFID tags or smart labels (defined below) are captured by the reader 60 via radio waves, PCMCIA smart cards, radio frequency cards, memory sticks, CDs, DVDs, or any other analog or digital storage media. Other examples of data input device 60 include a voice activation or recognition device or a portable personal data assistant (PDA). Depending upon the types of interface devices used, user interface device 54 and data input device 60 may be the same device. Although data input device 60 is shown as disposed within control unit 14, it is recognized that data input device 60 may be associated with a pharmacy system or located externally and communicating with pharmacy system through an appropriate communication means. Auxiliary interface 62 may be an RS-232 communications interface, however any other means for communicating with a peripheral device such as a printer, patient monitor, infusion pump or other medical device may be used without departing from the subject technology. Additionally, data input device 60 may be a separate functional module, such as modules 16, 18, 20 and 22, and configured to communicate with controller 14, or any other system on the network, using suitable programming and communication protocols.

According to various implementations, the patient care unit 12 further includes a network controller 52 for connecting the PCU and its various components to a network 10. Controller 52 may include a network interface that may be a wired or wireless connection, such as by Ethernet, WiFi, BLUETOOTH, an integrated services digital network (ISDN) connection, a digital subscriber line (DSL) modem or a cable modem. Any direct or indirect network connection may be used, including, but not limited to a telephone modem, an MIB system, an RS232 interface, an auxiliary interface, an optical link, an infrared link, a radio frequency link, a microwave link or a WLANS connection or other wireless connection. While the network controller 52 is depicted within the control unit 14, the controller 52 may reside in a functional module 16, 18, 20, 22 or in an external device (e.g., attached or proximate to PCU 12).

Functional modules 16, 18, 20, 22 are any devices for providing care to a patient or for monitoring patient condition. As shown in FIG. 1B, at least one of functional modules 16, 18, 20, 22 may be an infusion pump module such as an intravenous infusion pump for delivering medication or other fluid to a patient. Each of functional modules 16, 18, 20, 22 may be any patient treatment or monitoring device including, but not limited to, an infusion pump, a syringe pump, a PCA pump, an epidural pump, an enteral pump, a blood pressure monitor, a pulse oximeter, an EKG monitor, an EEG monitor, a heart rate monitor, an intracranial pressure monitor, or the like. Functional module 16, 18, 20 and/or 22 may also include a printer, scanner, bar code reader, near-field communication reader, RFID reader, or any other peripheral input, output or input/output device.

Each functional module 16, 18, 20 and/or 22 communicates directly or indirectly with control unit 14, with control unit 14 providing overall monitoring and control of device 12. Functional modules 16, 18, 20 and/or 22 may be connected physically and electronically in serial fashion to one or both ends of control unit 14 as shown in FIG. 1B. However, it is recognized that there are other means for connecting functional modules with the interface unit that may be utilized without departing from the subject technology. It will also be appreciated that devices such as pumps or patient monitoring devices that provide sufficient programmability and connectivity may be capable of operating as stand-alone devices and may communicate directly with the network without connected through a separate interface unit or control unit 14. As described above, additional medical devices or peripheral devices may be connected to patient care unit 12 through one or more auxiliary interfaces 62.

Each functional module 16, 18, 20, 22 may include module-specific operating systems, including components 76, a microprocessor 70, a volatile memory 72 and a nonvolatile memory 74 for storing information. It should be noted that while four functional modules are shown in FIG. 1A, any number of devices may be connected directly or indirectly to central controller 14. The number and type of functional modules described herein are intended to be illustrative, and in no way limit the scope of the subject technology. Module-specific components 76 include any components necessary for operation of a particular module, such as a pumping mechanism for infusion pump module 16.

While each functional module may be capable of at least some level of independent operation, control unit 14 monitors and controls overall operation of device 12. For example, as will be described in more detail below, control unit 14 provides programming instructions to the functional modules 16, 18, 20, 22 and monitors the status of each module.

Medical devices incorporating aspects of the subject technology may be equipped with a network interface module (NIM), allowing the medical device to participate as a node in a network. While for purposes of clarity the subject technology will be described as operating in an Ethernet network environment using the Internet Protocol (IP), it is understood that concepts of the subject technology are equally applicable in other network environments, and such environments are intended to be within the scope of the subject technology.

Data to and from the various data sources can be converted into network-compatible data with existing technology, and movement of the information between the medical device and network can be accomplished by a variety of means. For example, patient care unit 12 and network 10 may communicate via automated interaction, manual interaction or a combination of both automated and manual interaction. Automated interaction may be continuous or intermittent and may occur through direct network connection 52, or through RS232 links, MIB systems, RF links such as BLUETOOTH, IR links, WLANS, digital cable systems, telephone modems or other wired or wireless communication means. Manual interaction between patient care unit 12 and network 10 involves physically transferring, intermittently or periodically, data between systems using, for example, user interface device 54, coded data input device 60, bar codes, computer disks, portable data assistants, memory cards, or any other media for storing data. The communication means in various aspects is bidirectional with access to data from as many points of the distributed data sources as possible. Decision-making can occur at a variety of places within network 10. For example, and not by way of limitation, decisions can be made in an information system server 30 (e.g., health information system (HIS) server, decision support, or remote data server), hospital department or unit stations 32, or within patient care unit 12 itself.

All direct communications with medical devices operating on a network in accordance with the subject technology may be performed through server 30. In accordance with aspects of the subject technology, network interface modules incorporated into medical devices such as, for example, infusion pumps or vital signs measurement devices, may ignore all network traffic that does not originate from an authenticated user and/or system. In some implementations, server 30 may coordinate tracking of the location and status of all networked medical devices that have NIMs, and maintain open communication.

According to various implementations, server 30 may include a formulary and/or pharmacy information system. Pharmacy information systems may enable a safer physician medication order process. A pharmacy website (e.g., provided by the server) may provide the physician with a list of available drugs from which the physician may select (stored, e.g., in a database 37). The pharmacy website may contain a drug library having the list of available drugs but may also contain and present to the physician the drug names associated with recommended dosages and dose limits that have been established or adopted by the healthcare facility. In such a case where the physician need only select items from the computer screen rather than having to manually type in drug names and drug administration numbers (such as infusion rates, times, etc.) associated with administration of the medication, a more accurate medication process should result.

If a clinical order is for administration of a particular medication regimen, the order will be transmitted to the facility's pharmacy information server 30. The pharmacy reviews the order, and once the order has been prepared, the order may be transmitted to the nurse station (e.g., at workstation 32) for matching with the appropriate patient. Formulary is an approved list of drugs for use (e.g., available to order for a patient) within a medical facility. Within a formulary, there may be indication for use information and/or concentrations and drug ranges approved for the facility. As will be described further, a formulary may be used to define one or more medical device drug libraries, which may then be provided to infusion pumps within a hospital network. Inside the library, there is medication information such as drug names, concentration, diluent volume, strength, minimum or maximum infusion parameters for a drug, and other parameters. The formulary's establishment of these parameters, along with parameters for off-formulary orders, via the server 30 is useful for maintaining consistency across the healthcare environment and ensuring an order is intelligible and executed according to expectations by other devices within the system (e.g., an infusion pump).

With further reference to FIG. 1A, patient care unit 12 is capable of operating in several different modes, or personalities, with each personality defined by a configuration database. The configuration database may be a database 56 internal to patient care unit, or an external database 37. A particular configuration database is selected based, at least in part, by patient-specific information such as patient location, age, physical characteristics, or medical characteristics. Medical characteristics include, but are not limited to, patient diagnosis, treatment prescription, medical history, medical records, patient care provider identification, physiological characteristics or psychological characteristics. As used herein, patient-specific information also includes care provider information (e.g., physician identification) or a patient care unit's 12 location in the hospital or hospital computer network. Patient care information may be entered through interface device 52, 54, 60 or 62, and may originate from anywhere in network 10, such as, for example, from a pharmacy server, admissions server, laboratory server, and the like.

A memory 56, 58 of the control unit 14 may contain a drug library or libraries, an event log or logs, and pump configuration settings, such as, but not limited to, profiles to be used in particular practice areas such as ICU, PED, etc. The memory may be electronically loadable memory such as non-volatile memory (e.g., EEPROM). Drug libraries stored on pumps (which illustratively contain such information as the drug names, ranges of delivery parameter values such as proper concentrations, dosage units, and dose limits) can be used to perform drug calculation-based infusions in a clinical setting.

A drug library may be loaded from within the pump's memory and may include clinical order settings such as limits set by the clinical institution for each drug of the library (also termed as “guardrails” herein). Such limits may take the form of maximum and minimum dosages for each drug which may be made dependent on patient factors or other factors associated with delivery of the drug. For example, the dosage limits may vary depending on the weight of the patient or body surface area (“BSA”), depending on the unit or ward of the medical institution in which the drug is being used (for example neonatal care unit (NCU), the intensive care unit (ICU), etc.), and depending on other factors. An alarm may be provided if the nurse sets the pump to operate outside the range between the limits for a particular drug. In some cases, the alarm may be overridden and in other cases it may not. The medical facility may establish “soft” limits for each drug, which may be overridden by the nurse, and “hard” limits which may not. In either case where a limit is exceeded, a pump data log or other processor in communication with the infusion pump may record each such limit event for later analysis where the attempted setting is higher than the maximum or lower than the minimum dosage.

The pump also includes a display (described below) for displaying a user interface, including a control panel through which the user can navigate a workflow to program the PCU, control unit 14 and/or module 12-22, and select a drug library and/or drug entries from the drug library. Each of the associated sets of drug delivery parameters includes information selected from a group of parameters including drug concentration, drug delivery rate, drug dose, and bolus size. An electronically loaded drug library contains a list of available mode options specifying the units available for expressing drug delivery information, and the drug infusion pump offers the user the list of available mode options from which to make a selection when the electronically loaded drug library is in the pump. In the case of a syringe pump, the electronically loaded drug library may include a list of names of syringe manufacturers identifying syringes that can be used in the drug infusion pump, and the drug infusion pump offers the user the list of names of syringe manufacturers from which to make a selection when the electronically loaded drug library is in the pump. The loaded drug library may include a list of syringe sizes identifying syringes that can be used in the drug infusion pump, and the drug infusion pump offers the user the list of syringe sizes from which to make a selection when the electronically loaded drug library is in said pump. In the case of a peristaltic pump, the electronically loaded drug library may include a list of infusion set manufacturers. A loaded drug library may include a set of features, each of which is either toggled on or off, and the pump offers the user only the features from among the set of features that are toggled on when the electronically loaded drug library is in said pump.

FIG. 1B is another view of a portion of the example patient care unit 12 shown in FIG. 1A, according to aspects of the subject technology. FIG. 1B shows two fluid infusion pump modules 18, 20 mounted at either side of a control unit 14, and the displays and control keys of each, with the control unit 14 being capable of programming both infusion pump modules 18, 20. While the pump modules 18, 20 are shown as distinct devices that are separable from the control unit 14, it is understood that some implementations may include a single integrated control unit and pump system.

An infusion pump module includes a door 5a and a handle 5b that operates to lock the door in a closed position for operation and to unlock and open the door for access to the internal pumping and sensing mechanisms and to load administration sets for the pump module. When the door 5a is open, the tube can be connected with the pump. When the door 5a is closed, the tube is brought into operating engagement with the pumping mechanism, the upstream and downstream pressure sensors, and the other equipment of the pump. A display 5c, such as an LED display, is located in plain view on the door in this embodiment and may be used to visually communicate various information relevant to the pump, such as alert indications (e.g., alarm messages). Control keys 5e-h exist for programming and controlling operations of the respective infusion pump module 18, 20. In some implementations, the control keys may be presented as interactive elements on the display 5c (e.g., touchscreen display). The infusion device and/or infusion pump may also include audio alert equipment in the form of a speaker (not shown).

The control unit 14 of the infusion device 12 includes a display 6a for visually communicating various information, such as the operating parameters of a connected pump module and alert indications and alert messages, and control keys 6b and 6c for selecting and/or setting control parameters and/or options for controlling the infusion device 12 and connected modules.

Control keys 6 may exist for programming and controlling operations of the infusion pump module as desired including, for example, for navigating steps of a predetermined workflow to enter parameters and select a drug library. The control unit 14 may also include a speaker to provide audible alerts. In some implementations, the display 6a may be implemented as a touchscreen display. In such implementations, the control keys 6b may be omitted or reduced in number by providing corresponding interactive elements via a graphical user interface presented via the display 6a. In some implementations, each control key 6b (or 6c) may select a corresponding option displayed in display 6b.

The control unit 14 may include one or more communications systems (as described with regard to FIG. 1A) with which the control unit 14 may communicate with external equipment such as a medical facility server or other computer and with a portable processor, such as a handheld communication device or a laptop-type of computer, or other information device that a clinician may have to transfer information as well as to download drug libraries to a programming module 16, 18, 20, 22 (such as pump 20). A communication module may be used to transfer access and interaction information for clinicians encountering the programming module or device coupled therewith (e.g., pump 20 or bar code scanner). As described previously, the communications system may include one or more of a radio frequency (RF) system, an optical system such as infrared, a BLUETOOTH™ system, or other wired or wireless system. The bar code scanner and communications system may alternatively be included integrally with the infusion pump 20, such as in cases where a programming module is not used, or in addition to one with the control unit 14. Further, information input devices need not be hard-wired to medical instruments, information may be transferred through a wireless connection as well. Additionally, other types of modules may be connected to the pump modules or to the programming module such as a syringe pump module, patient controlled analgesic module, End Tidal CO₂ monitoring module, oximeter monitoring module, or the like.

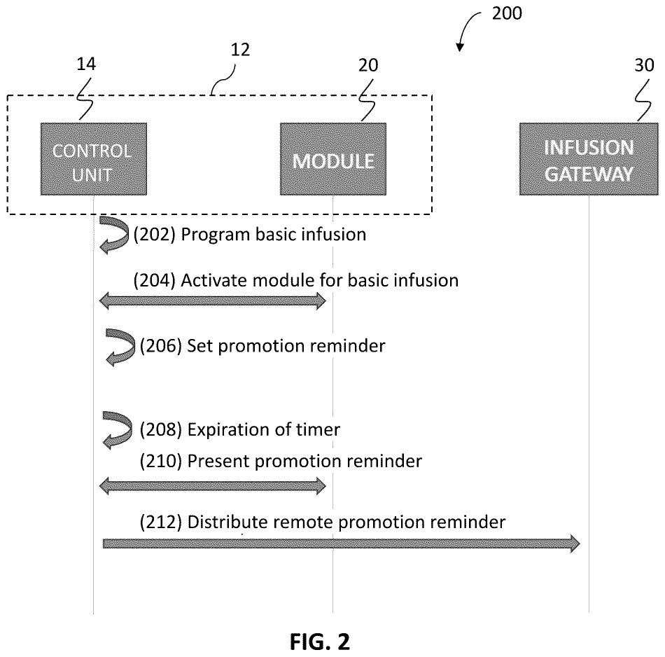

FIG. 2 depicts an example sequence diagram 200 of providing a infusion promotion reminder for rapid infusion workflow promotion, according to various aspects of the subject technology. In the depicted example, an infusion device PCU 12 is programmed for a basic infusion (202). According to various implementations, a basic infusion is an infusion that is initiated with only a minimal set of parameters. Programming of the infusion device and/or its modes may occur by way of a clinician manipulating one or more input controls on a control unit 14 to program an infusion pump module 20 to administer a medication to a patient. For a basic mode, a clinician may only enter a flow rate or volume to be infused (VTBI), without any indication of the patient, characteristics of the patient, or what time type of medication is being infused. The module is then activated (204) and the infusion pump on the programmed module 20 begins infusing according to the basic programming it received.

According to various implementations, on initiating and start of the basic mode, the infusion device 12 generates a timer for setting up a promotion reminder (206) by which the infusion device will issue a notification to remind the clinician, or another clinician, that the infusion device has not yet been set up in its preferred mode for infusion and that further programming should be performed to enter a preferred mode of operation. Responsive to an expiration of the timer (208), the infusion device automatically causes a presentation of the promotion reminder (210). The presentation may occur on a display device of the control unit 14 and/or on the respective infusion pump module 20. In some implementations, the promotion reminder may include a display of a graphical window that overlays and prevents interaction with at least a portion of the user interface on the display device.

As will be described further, presenting the reminder may include a presentation of a control (e.g., a virtual control) configured to, when activated, reduce the number of workflow steps that are required to identify the type of the infusion and to switch the infusion device from the initial to preferred mode. For example, selecting a type of the infusion may involve a predetermined number of steps to navigate a number of selections and display screens to reach a designated interface/screen for selecting the type of the infusion. Activation of the control may bypass these selections and/or screens and immediately present the selection screen to the user (thereby reducing the number of steps). In this regard, the reminder may include activating or associating one or more control elements (e.g., button or softkeys) with a promote option to migrate the existing infusion to a new mode in which guardrails (e.g., predetermined limits for a particular drug) or a drug library is used. In the event that a basic infusion was initiated because drug was missing from the library, the promotion reminder may include an option to set a “channel label” which at least receives a drug name to show on the pump, which can then be reported to an electronic medical record server (e.g., server 30).

In some implementations, an indication of the promotion reminder may be distributed (e.g., sent) to an infusion gateway 30 (212) for either reporting purposes or for notification to mobile devices. For example, clinician may register for notifications pertaining to infusion devices in the clinician's care unit or area of responsibility. When a timer expires on a respective device then the device may send a message to the infusion gateway to notify all clinicians that are registered to receive notifications for the device and/or area associated with the device. In some implementations, statistics pertaining to the reporting and/or promotions may be sent to the server 30 and used to determine when reminders have appeared for a device and/or to determine how long it takes for a device to be promoted.

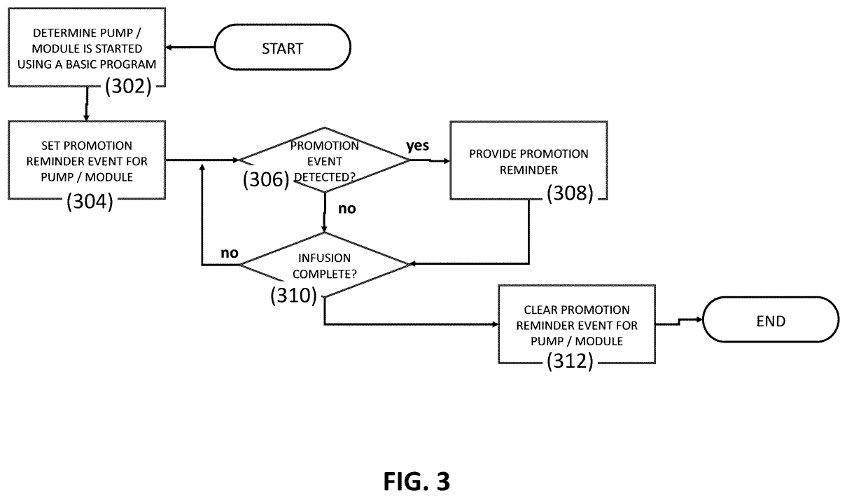

FIG. 3 depicts an example process flow diagram 300 for providing a infusion promotion reminder for rapid infusion workflow promotion, according to aspects of the subject technology. For explanatory purposes, the various blocks of example process 300 are described herein with reference to FIGS. 1-2, and the associated components and/or processes described herein. The one or more of the blocks of process 300 may be implemented, for example, by a PCU 12 or associated module 16, 18, 20, 22 or control unit 14, or processor thereof. In some implementations, one or more of the blocks may be implemented, at least in part, based on one or more machine learning algorithms. In some implementations, one or more of the blocks may be implemented apart from other blocks, and by one or more different processors or devices. Further, for explanatory purposes, to the extent that the blocks of example process 300 are described as occurring in serial, or linearly, in some implementations, multiple blocks of example process 300 may occur in parallel. In addition, the blocks of example process 300 need not be performed in the order shown and/or one or more of the blocks of example process 300 need not be performed.

In the depicted example, the system determines that a pump module (e.g., a module 18, 20) is started in a first operational mode using a basic infusion program (302). As described previously, this first mode may be initiated merely by entering a flow rate and starting the pump. A promotion reminder event (e.g., a trigger condition) is then set for the module (304). According to some implementations, the event includes the expiration of a timer that is set on start of the infusion. In some implementations, the event may include a shift change associated with the care area of the pump or a characteristic of the pump itself. For example, the event may include a predetermined percentage of the fluid being delivered (e.g., when VTBI is entered), or a certain amount of fluid being delivered (e.g., over 5 mL). The event may include the next time a clinician (same or different) badges in (e.g., is authenticated) to the PCU, or when an alarm associated with the PCU is being cleared (e.g., during a pause), or when it can be determined that a clinician is focused on the pump (e.g., by detection of key press, sensor based proximity, and the like). As will be described further, when the event includes a timer, the length of the timer may be adjusted by any one of these factors.

The infusion device (or associated computing device) monitors for the promotion event (306) and, when detected, provides a promotion reminder (308) as previously described. Whether or not detected, the process continues until the infusion is complete (310). In this regard, according to some implementations, if the event is detected and a reminder dismissed then the event may be reset so that another reminder is generated on the next occurrence of the event. Once the infusion is determined to be completed (e.g., by the VTBI being reached or the pump detecting there is no more fluid to be infused), the promotion reminder event may be cleared (312) so that future reminders will not be presented.

Technical factors may include conditions and events associated with the infusion pump, while clinical factors may include conditions and events associated with a patient receiving a therapy from the infusion pump, the therapy, or a care area associated with the patient. For example, a higher rate or more critical care unit (e.g., NICU) may shorten the length of the timer (timeout interval) so that reminders are issued sooner. The timeout interval may be determined by a processor 50 of the control unit 14 or, in some implementations, by a processor/microprocessor 70 of a functional module. The information utilized to determine the timeout interval may be retrieved from external systems, for example, from an internal data store 56 or an external data store 37 or external server or other operatively connected device (e.g., server 30 or remote terminal 32). In some implementations, the timeout interval may be set by way of user input via a user interface. In some implementations, the information utilized may be derived from operational data, parameters, and/or measurements from integrated systems.

Technical factors may include, for example, how many functional modules are currently attached to a control unit 14. In some implementations, a processor may determine the number of functional modules connected to the control unit. The determination may be by interrogation of the connection ports or based on stored data in memory of the control unit 14 indicating how many devices (and what type) are connected. The processor may then determine the timeout interval based on the number of functional modules. In this regard, the duration of the timeout interval may decrease inversely according to an increase in the number of functional modules. For example, the timeout may be reduced to 10 m from 20 m in response to the number of connected modules increasing from three to four connected modules.

In some implementations, the processor may detect that the PCU (or connected module) was switched from a fixed power source (e.g., AC power) to a battery (e.g., DC power) and, in response, increase the timeout interval to a predetermined interval that is greater than the current timeout interval to conserve power. In some implementations, the processor may monitor the capacity of the battery over time and, responsive to the battery reaching a threshold (reduced) capacity, the timeout interval may be reduced to a second predetermined interval that is less than the current predetermined interval.

In some implementations, sensors within the PCU or a functional module may detect that an infusion pump is undergoing a loading event (e.g., a syringe is being inserted into a syringe pump or an IV infusion set is being loaded into a pumping mechanism). The loading even may trigger the promotion event (306).

In some implementations, clinical factors affecting the timeout interval may include the number of clinicians currently staffed in a care area associated with the PCU. The timeout interval may be set to a first threshold interval responsive to the number of clinicians satisfying a first threshold number and may be set to a second threshold interval responsive to the number of clinicians satisfying a second threshold number. In some implementations, the threshold may be set based on patient acuity. In this regard, the timeout interval may be reduced when the patient associated with the PCU is identified as being in a high-risk category. A high-risk category may be inferred by the assigned care unit. For example, if the patient associated with the infusion pump, or the infusion pump itself, is associated with a high-risk care area (e.g., the ICU or NICU) then the timeout may be adjusted accordingly (e.g., reduced). In some implementation, the timeout interval may be adjusted or set based on the fluid being infused by the infusion pump. For example, controlled substances may be associated with a higher frequency of status messages and/or lower timeout intervals. In this regard, the timeout interval may be reduced when the therapy is a medication identified as a predetermined controlled substance.

In some implementations, the duration of the timeout interval may be reduced—or the promotion event triggered—in response to the PCU, functional module, or associated component, experiencing an alarm condition. The processor may detect, within a predetermined window of time, one or more alarm conditions associated with a therapy being administered by the device and trigger the promotion event or determine a new reduced timeout interval.

According to various implementations, the PCU 14 or a computing device associated with the PCU (e.g., a connected mobile device or terminal 32) may provide a user interface for setting the timeout interval/trigger condition and/or adjusting how a timeout interval is determined based on the at least one predetermined technical factor or the at least one clinical factor. The interface may include, for example, one or more controls that enable a user to set whether the timeout interval is increased or decreased based on one or more predetermined conditions. For example, a user may set whether the timeout interval is increased or decreased when power is switched to the battery, and/or whether the timeout interval is increased or decreased when the power level of the battery reaches a threshold capacity (e.g., 4 Ah). The same or similar selections may be made for operating parameters (e.g., volume infused, etc.) or sensor readings (e.g., temperature reaches a certain threshold, an occlusion is detected, etc.).

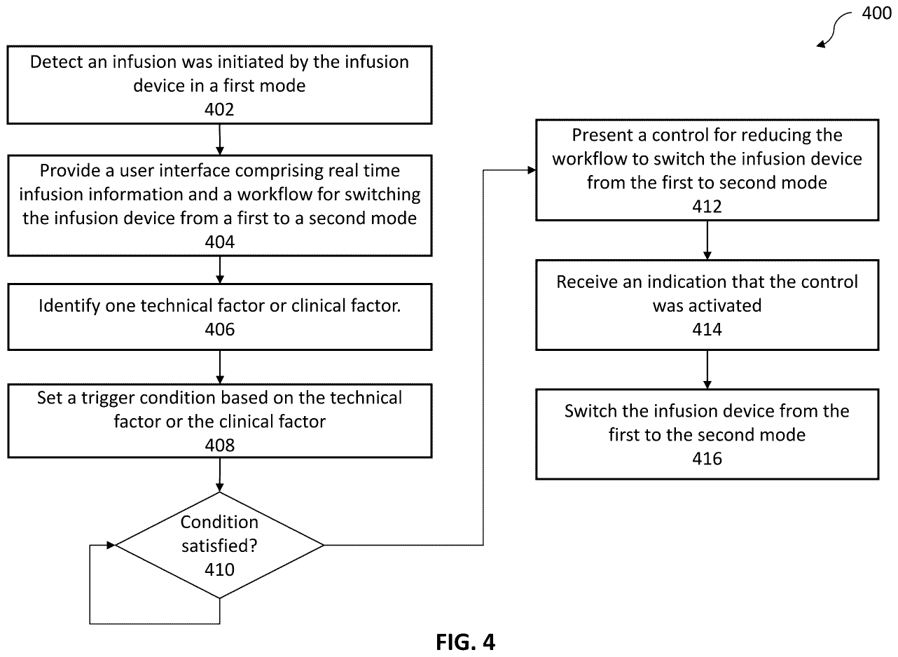

FIG. 4 depicts an example process flow diagram 400 for rapid infusion workflow promotion, according to aspects of the subject technology. For explanatory purposes, the various blocks of example process 300 are described herein with reference to FIGS. 1-2, and the associated components and/or processes described herein. The one or more of the blocks of process 300 may be implemented, for example, by a PCU 12 or associated module 16, 18, 20, 22 or control unit 14, or processor thereof (generally, the “system”). In some implementations, one or more of the blocks may be implemented, at least in part, based on one or more machine learning algorithms. In some implementations, one or more of the blocks may be implemented apart from other blocks, and by one or more different processors or devices. Further, for explanatory purposes, to the extent that the blocks of example process 300 are described as occurring in serial, or linearly, in some implementations, multiple blocks of example process 300 may occur in parallel (e.g., blocks 404 and 406 may be in parallel). In addition, the blocks of example process 300 need not be performed in the order shown and/or one or more of the blocks of example process 300 need not be performed.

In the depicted example, the disclosed system detects that an infusion was initiated by the infusion device in a first mode (402). According to various implementations, when the infusion device is in the first mode, a type of the infusion (and/or the identity of the patient) is unknown to the infusion device. As described previously, the first mode may be a default mode of the device or may be a mode in which minimal parameters are entered. When the type of infusion is unknown the infusion device may only receive a flow rate or a VTBI, and may have no knowledge of the patient, type of infusion, or medication being infused. In this regard, the system may determine that the device is in the first mode by way of specifically determining the absence of one or more (or specific number of) certain parameters (e.g., an identity of the patient and/or medication), or the absence of a drug library being used/loaded for the infusion.

The system causes, on a display device associated with the infusion device, a display of a user interface comprising real time information pertaining to the infusion (404). The display may include, for example, a display device 6a of the control unit 14 or a display device 5c the infusion pump module, or an external display associated with the infusion device (e.g., a multi-parameter monitor, nursing station monitor, or mobile device of a clinician). According to various implementations, the (default) user interface provides an interactive workflow including a number of workflow steps that are required to identify the type of the infusion being initiated, and for switching the infusion device from the first mode to a second mode that is based on the identified type of the infusion.

In some implementations, the number of workflow steps include a predetermined sequence of screens that each prompt a clinician for specific input to program the device. For example, to promote an infusion, the clinician may select the channel that is currently active in basic mode on a first screen, choose an option for activating a drug library on a second screen, then select the drug that is currently being infused from the library on a third screen, and then select to activate the library. The infusion device may then implement infusion limits for the particular drug based on the drug library and begin monitoring the infusion to determine whether the infusion parameters are outside of those limits, and alert accordingly. In the same regard, the clinician may also electronically associate the current infusion with a particular patient (or patient order) on another screen, and the infusion device may implement and/or adjust limits on the infusion for that particular patient.

In the depicted example, when the infusion device is initiated in the first mode (e.g., without activating the drug library or identifying the patient or medication), the system identifies at least one technical factor associated with the infusion device or at least one clinical factor associated with a patient receiving the infusion or a care area or clinician associated with the infusion device (406). A trigger condition is then set for presenting a prompt for switching the infusion device from the first mode to a second mode (408). The system then monitors for the occurrence of an event that satisfies the trigger condition (410).

According to various implementations, the clinical and/or technical factors are determined from predetermined clinical factors and predetermined technical factors. In some implementations, a clinical factor includes the specific type of care area associated with the infusion device or the patient. In some implementations, the criticality of the care area/ward is associated with the timer length/value (e.g., the timer interval). For example, more critical patients are associated with more critical drugs that are more dangerous at unsafe rates. Allowing a drug to be infused without specific guardrails for the drug (e.g., from a drug library) can have dangerous consequences if left unchecked. Accordingly, the more critical the care unit or patient, the shorter the length of time that the system allows an infusion to continue without being promoted (e.g., the system may not allow an infusion to run more than 5 minutes without a promotion reminder).

The system may be further configured to receive an indication that the infusion device is assigned to a care area associated with high risk patients (e.g., NICU or ICU) and set the trigger condition by initiating a predetermined timer associated with the risk. The indication of the care unit association may be by way of prior programming or by way of cross reference of an identifier of the infusion pump with a care unit in a data store. In some implementations, the timer may be set by performing a lookup in a database based on the identity of the care unit to obtain a specific timer length/value corresponding to the care unit or care unit type.

In some implementations, a clinical factor includes a scheduled shift change within the care area that affects a clinician associated with the infusion device. In this regard, the system may be further configured to identify the care area associated with the infusion device, determine an occurrence of the shift change within the identified care area that affects the clinician(s) associated with the infusion device (e.g., based on current time), and set the event by initiating a timer responsive to the occurrence of the shift change. The occurrence of the shift change may be determined based on a predetermined schedule and/or other inputs received at the corresponding hospital information server 30 associated with the system.

In some implementations, a clinical factor includes the infusion device receiving input of an atypical parameter value. In this regard, the system may be further configured to receive an operational parameter for administering the infusion, determining that a value of the received operational parameter is atypical of historical values for the operational parameter, and set the event by initiating a timer responsive to determining that the value of the received operational parameter is atypical of historical values for the operational parameter. Whether a parameter value is atypical may be based on machine learning, averaging, historical algorithm, or other calculations. A historical algorithm may, for example, look for divisions of a predetermined number such as 2, 5, or 10 (e.g., depending on the care area and/or preprogramming) and determine a parameter is atypical if not divisible by the number. A historical algorithm may also determine whether a value entered is atypical for the particular care area associated with the infusion device. For example, if a flow rate of 210 or 221 ml/hr is entered and a value of 21 ml/hr is typical for the care area then the entered value may be flagged as atypical.

In some implementations, the input of the atypical parameter value includes an atypical infusion rate for the infusion device. In this regard, the system may receive an input of the rate of the infusion, determine that the rate of the infusion satisfies a predetermined threshold rate or a rate that is atypical of historical rates used with the infusion device, and set the event by initiating a timer responsive to determining that the rate satisfies the threshold or is otherwise atypical. In some implementations, a technical factor includes the infusion rate being atypical for an infusion source loaded into the infusion device. In this regard, the system may be configured to receive an indication of a type of the infusion source loaded into the infusion device, receive an input of the infusion rate, determine that the infusion rate is atypical for the type of the infusion source loaded into the infusion device (e.g., based on machine learning, averaging, or other calculations); and set the event by initiating a timer responsive to determining that the infusion rate is atypical for the type of the infusion source loaded into the infusion device.

When the infusion device includes a syringe pump, the system may determine a size of the syringe loaded into the infusion device (e.g., based on a sensor integrated in the pump), and determine that the set infusion rate is above a threshold rate that is predetermined for the size of the syringe (e.g., based on a lookup of the size and/or syringe type in a lookup table).

Continuing with the process 400 of FIG. 4, responsive to detection of the event, a prompt and a control is presented for reducing the workflow to switch the infusion device from the first mode to the second mode (412). In some implementations, the prompt and control is presented in the form of a banner, icon, or overlay within the current user interface (which, e.g., is displaying real time infusion information). In some implementations, the prompt and/or control includes a pop up window. In some implementations, the prompt and control may include a full page replacement screen for a screen that is currently displayed in the user interface (e.g., replacing or supplementing the infusion information for the current infusion). Accordingly, the prompt may force the user to either select the control or provide a reason to override. If the prompt is closed then the system may require input of a reason to override the mode promotion, which then may be logged for future reporting purposes.

Additionally or in the alternative, the prompt may include an audio and/or visual alert. In some implementations, the system automatically causes, on the display device, a display of a graphical window that overlays and prevents interaction with at least a portion of the user interface on the display device.

In some implementations, the control is a virtual control that may be displayed, for example, on the display device. The virtual control may be activated by a touch input received at the control on the touch sensitive display or by way of a physical control 6b (FIG. 1B) associated with the display of the virtual control.

According to various implementations, the control is configured to, when activated, reduce the predetermined number of workflow steps that are required to identify the type of the infusion and to switch the infusion device from the first mode to a second mode. For example, activating the control may take the user directly to the drug library (or medication) selection screen, thereby bypassing the channel selection screen and other option screens (as the prompt and/or control are already associated with the particular options that require promoting).

In some implementations, the trigger condition (e.g., the expiration of a timer) can be bypassed on the occurrence of certain predetermined events, so that prompt and/or control is immediately presented. The system may, for example, receive an indication of a user authentication to the infusion device, and bypass the timer to cause the display of the prompt (including, e.g., the graphical window) responsive to the indication of the user authentication. In some implementation, wherein the infusion is initiated on a first channel of the infusion device, the system may be configured to, when an indication is received that another infusion on a second channel of the infusion device was initiated in or updated to the second mode, the trigger condition is bypassed to cause a display of the prompt responsive to the other infusion being initiated in or updated to the second mode.

Further continuing the process 400 of FIG. 4, the system receives an indication that the control is activated (414). In some implementations, the indication may include an indication that the type of the infusion was identified via the reduced number of workflow steps being completed by a user.

In some implementations, the graphical window is displayed on a mobile device assigned to a clinician associated with the infusion device. In such implementations, the activation of the control and completion of the workflow steps may be received at the mobile device (and transmitted to the infusion device via a network).

The system then switches the infusion device from the first mode to the second mode (416). According to various implementations, the switching is based on the type of the infusion being identified and the reduced number of workflow steps being completed by the user. In this regard, the infusion may be subject to limits and/or guardrails imposed by a drug library or other monitoring controls set by the infusion pump when in the second mode.

Many of the above-described example processes 300 and 400 and related features and applications, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

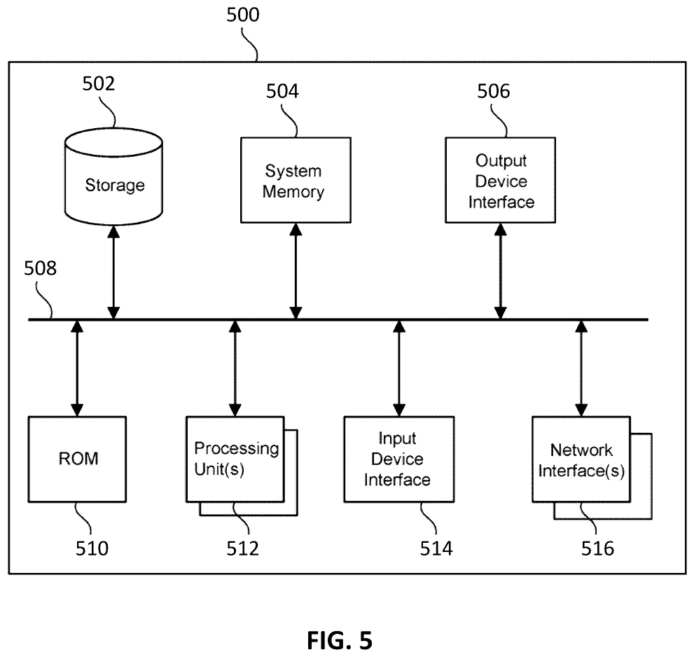

FIG. 5 is a conceptual diagram illustrating an example electronic system 500 for rapid infusion workflow promotion, according to aspects of the subject technology. Electronic system 500 may be a computing device for execution of software associated with one or more portions or steps of processes 300, 400 or components and methods provided by FIGS. 1-4, including but not limited to computing hardware within PCU 12, control unit 14, infusion module(s) 16-22, server 30, computing device 32, and/or any computing devices or associated modules or terminals disclosed herein. Electronic system 500 may be a personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Electronic system 500 may include various types of computer readable media and interfaces for various other types of computer readable media. In the depicted example, electronic system 500 includes a bus 508, processing unit(s) 512, a system memory 504, a read-only memory (ROM) 510, a permanent storage device 502, an input device interface 514, an output device interface 506, and one or more network interfaces 516. In some implementations, electronic system 500 may include or be integrated with other computing devices or circuitry for operation of the various components and methods previously described.

Bus 508 collectively represents all system, peripheral, and chipset buses that communicatively connect the numerous internal devices of electronic system 500. For instance, bus 508 communicatively connects processing unit(s) 512 with ROM 510, system memory 504, and permanent storage device 502.

From these various memory units, processing unit(s) 512 retrieves instructions to execute and data to process, in order to execute the processes of the subject disclosure. The processing unit(s) can be a single processor or a multi-core processor in different implementations.

ROM 510 stores static data and instructions that are needed by processing unit(s) 512 and other modules of the electronic system. Permanent storage device 502, on the other hand, is a read-and-write memory device. This device is a non-volatile memory unit that stores instructions and data even when electronic system 500 is off. Some implementations of the subject disclosure use a mass-storage device (such as a magnetic or optical disk and its corresponding disk drive) as permanent storage device 502.

Other implementations use a removable storage device (such as a floppy disk, flash drive, and its corresponding disk drive) as permanent storage device 502. Like permanent storage device 502, system memory 504 is a read-and-write memory device. However, unlike storage device 502, system memory 504 is a volatile read-and-write memory, such as, random access memory. System memory 504 stores some of the instructions and data that the processor needs at runtime. In some implementations, the processes of the subject disclosure are stored in system memory 504, permanent storage device 502, and/or ROM 510. From these various memory units, processing unit(s) 512 retrieves instructions to execute and data to process in order to execute the processes of some implementations.

Bus 508 also connects to input and output device interfaces 514 and 506. Input device interface 514 enables the user to communicate information and select commands to the electronic system. Input devices used with input device interface 514 include, e.g., alphanumeric keyboards and pointing devices (also called “cursor control devices”). Output device interfaces 506 enables, e.g., the display of images generated by the electronic system 500. Output devices used with output device interface 506 include, e.g., printers and display devices, such as cathode ray tubes (CRT) or liquid crystal displays (LCD). Some implementations include devices such as a touchscreen that functions as both input and output devices.

Also, as shown in FIG. 5, bus 508 also couples electronic system 500 to a network (not shown) through network interfaces 516. Network interfaces 516 may include, e.g., a wireless access point (e.g., Bluetooth or WiFi) or radio circuitry for connecting to a wireless access point. Network interfaces 516 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 500 can be used in conjunction with the subject disclosure.

These functions described above can be implemented in computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

### Illustration of Subject Technology as Clauses:

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identification.

Clause 1. An infusion device configured to: detect an infusion was initiated by the infusion device in a first mode, wherein when the infusion device is in the first mode a type of the infusion is unknown to the infusion device; cause, on a display device associated with the infusion device, a display of a user interface comprising real time information pertaining to the infusion, the user interface providing an interactive workflow comprising a predetermined number of workflow steps that are required to identify the type of the infusion and to switch the infusion device from the first mode to a second mode that is based on the identified type of the infusion; when the infusion is initiated in the first mode: identify at least one technical factor associated with the infusion device or at least one clinical factor associated with a patient receiving the infusion or a care area or clinician associated with the infusion device; and generate a trigger condition based on the identified at least one technical factor or the at least one clinical factor; responsive to the trigger condition being satisfied, automatically cause, on the display device, a display of a graphical window that overlays and prevents interaction with at least a portion of the user interface on the display device, and which presents a control configured to, when activated, reduce the predetermined number of workflow steps that are required to identify the type of the infusion and to switch the infusion device from the first mode to a second mode; receive, after the graphical window is displayed, an indication that the control was activated and that the type of the infusion was identified via the reduced number of workflow steps being completed by a user; and switch the infusion device from the first mode to the second mode based on the type of the infusion and the reduced number of workflow steps being completed by the user.

Clause 1. An infusion device configured to: detect an infusion was initiated by the infusion device in a first mode, wherein when the infusion device is in the first mode a type of the infusion is unknown to the infusion device; cause, on a display device associated with the infusion device, a display of a user interface comprising real time information pertaining to the infusion, the user interface providing an interactive workflow comprising a number of workflow steps that are required to select the type of the infusion and to switch the infusion device from the first mode to a second mode that is based on the selected type of the infusion; when the infusion is initiated in the first mode: generate a trigger condition based on at least one technical factor associated with the infusion device or at least one clinical factor associated with a patient receiving the infusion or a care area or clinician associated with the infusion device; responsive to the trigger condition being satisfied, automatically cause, on the display device, a display of a graphical window that presents a control configured to, when activated, prompt a selection of the type of the infusion to switch the infusion device from the first mode to a second mode; receive, after the graphical window is displayed, an indication that the control was activated and that the type of the infusion was selected; and switch the infusion device from the first mode to the second mode based on the selected type of the infusion.

Clause 2. The infusion device of Clause 1 or Clause 2, wherein the infusion device is further configured to: identify, when the infusion is initiated in the first mode, the at least one technical factor or the at least one clinical factor; reduce, when the control is activated, the number of workflow steps that are required to select the type of the infusion and to switch the infusion device from the first mode to the second mode, wherein the infusion device is switched from the first mode to the second mode via the reduced number of workflow steps being completed by a user

Clause 3. The infusion device of Clause 1 or Clause 2, wherein the at least one clinical factor comprises the infusion device being assigned to a care area associated with high risk patients, the infusion device being further configured to: receive an indication that the infusion device is assigned to a care area associated with high risk patients; and initiate a timer responsive to the indication that the infusion device is assigned to the care area associated with high risk patients, wherein the trigger condition comprises an expiration of the timer.

Clause 4. The infusion device of Clause 1 or Clause 2, wherein the at least one clinical factor comprises a shift change within the care area that affects a clinician associated with the infusion device, the infusion device being further configured to: identify the care area associated with the infusion device; determine an occurrence of the shift change within the identified care area that affects the clinician associated with the infusion device; and initiate a timer responsive to the occurrence of the shift change, wherein the trigger condition comprises an expiration of the timer.

Clause 5. The infusion device of Clause 1 or Clause 2, wherein the at least one clinical factor comprises receiving input of an atypical parameter value, the infusion device being further configured to: receive an operational parameter for administering the infusion; determining that a value of the received operational parameter is atypical of historical values for the operational parameter; and initiate a timer responsive to determining that the value of the received operational parameter is atypical of historical values for the operational parameter, wherein the trigger condition comprises an expiration of the timer.

Clause 6. The infusion device of Clause 1 or Clause 2, wherein the at least one clinical factor comprises receiving input of an atypical infusion rate, the infusion device being further configured to: receive an input of a rate for the infusion; determine that the rate for the infusion satisfies a threshold rate; and initiate a timer responsive to determining that the rate for the infusion satisfies the threshold rate, wherein the trigger condition comprises an expiration of the timer.

Clause 7. The infusion device of Clause 1 or Clause 2, wherein the at least one technical factor comprises the infusion device being programmed for an infusion rate that is atypical for an infusion source loaded into the infusion device, the infusion device being further configured to: receive an indication of a type of the infusion source loaded into the infusion device; receive an input of the infusion rate; determine that the infusion rate is atypical for the type of the infusion source loaded into the infusion device; and initiate a timer responsive to determining that the infusion rate is atypical for the type of the infusion source loaded into the infusion device, wherein the trigger condition comprises an expiration of the timer.

Clause 8. The infusion device of Clause 7, wherein the infusion device comprises a syringe pump and the infusion source is a syringe, the infusion device further configured to: determine a size of the syringe loaded into the infusion device, wherein determining that the infusion rate is atypical for the type of the infusion source comprises determining that the infusion rate is above a threshold rate that is predetermined for the size of the syringe.

Clause 9. The infusion device of Clause 1 or Clause 2, wherein the trigger condition comprises an expiration of a timer, the infusion device being further configured to: receive an indication of a user authentication to the infusion device; and bypass the timer to cause the display of the graphical window responsive to the indication of the user authentication.

Clause 10. The infusion device of Clause 1 or Clause 2, wherein the trigger condition comprises an expiration of a timer, and wherein the infusion is initiated on a first channel of the infusion device, the infusion device being further configured to: receive an indication that another infusion on a second channel of the infusion device was initiated in or updated to the second mode; and bypass the timer to cause the graphical window to be displayed responsive to the indication that the other infusion was initiated in or updated to the second mode.

Clause 11. The infusion device of any one of Clauses 1-10, wherein causing the display of the graphical window comprises: causing the graphical window to be displayed on a mobile device assigned to a clinician associated with the infusion device, wherein activation of the control and completion of the workflow steps is received at the mobile device.

Clause 12. A machine-implemented method, comprising: detecting an infusion was initiated by an infusion device in a first mode, wherein when the infusion device is in the first mode a type of the infusion is unknown to the infusion device; responsive to the infusion device being initiated in the first mode, generating a trigger condition based on at least one technical factor associated with the infusion device or at least one clinical factor associated with a patient receiving the infusion or a care area or clinician associated with the infusion device; responsive to the trigger condition being satisfied, automatically causing, on a display device associated with the infusion device, a display of a control configured to, when activated, provide a user workflow to select the type of the infusion and to switch the infusion device from the first mode to a second mode; receiving, after the control is displayed, an indication that the control was activated and that the type of the infusion was selected via the user workflow being completed by a user; and switching the infusion device from the first mode to the second mode based on the selected type of the infusion.

Clause 13. A machine-implemented method of Clause 12, wherein the trigger condition comprises an expiration of a timer, the method further comprising: responsive to the expiration of the timer, causing the display device to display a prompt to switch the infusion device from the first mode to the second mode together with the control, and restricting use of the infusion device by the user until the prompt is acknowledged by the user.

Clause 14. A machine-implemented method of Clause 12, wherein the trigger condition comprises an expiration of a timer, and wherein the at least one clinical factor comprises: the infusion device being assigned to a care area associated with high risk patients, a shift change within the care area that affects a clinician associated with the infusion device, or receiving input of an atypical parameter value, wherein the method further comprises detecting an occurrence of the at least one clinical factor, wherein the timer is initiated responsive to the occurrence of the at least one clinical factor.

Clause 15. A machine-implemented method of Clause 12, wherein the trigger condition comprises an expiration of a timer, and wherein the at least one technical factor comprises detecting an atypical infusion source being loaded into the infusion device, wherein the method further comprises detecting an infusion source was loaded into the infusion device and that the infusion source is atypical for historical infusion sources associated with the infusion device.

Clause 16. The machine-implemented method of Clause 12, wherein the trigger condition comprises an expiration of a timer, the method further comprising: receiving, while the control is not displayed, an indication of a user authentication to the infusion device; and bypassing the timer to cause the display of the control responsive to the indication of the user authentication.

Clause 17. The machine-implemented method of Clause 12, wherein the trigger condition comprises an expiration of a timer, and wherein the infusion is initiated on a first channel of the infusion device, the infusion device being further configured to: receiving, while the control is not displayed, an indication that another infusion on a second channel of the infusion device was initiated in or updated to the second mode; and bypassing the timer to cause the control to be displayed responsive to the indication that the other infusion was initiated in or updated to the second mode.

Clause 18. The machine-implemented method of Clause 12, wherein causing the display of the control comprises: causing a graphical interface to be displayed on a mobile device assigned to a clinician associated with the infusion device, wherein activation of the control and completion of the user workflow is received at the mobile device.

Clause 19. A non-transitory machine-readable medium comprising instructions thereon that, when executed by one or more processors, cause the one or more processors to perform a method according to any one of Clauses 12-18.

Clause 19. A machine-implemented method, comprising: detecting an infusion was initiated by an infusion device in a first mode, wherein when the infusion device is in the first mode a type of the infusion is unknown to the infusion device; causing, on a display device associated with the infusion device, a display of a user interface comprising real time information pertaining to the infusion, the user interface providing an interactive workflow comprising a number of workflow steps that are required to identify the type of the infusion and to switch the infusion device from the first mode to a second mode that is based on the identified type of the infusion; when the infusion is initiated in the first mode: identifying at least one technical factor associated with the infusion device or at least one clinical factor associated with a patient receiving the infusion or a care area or clinician associated with the infusion device; and generating a trigger condition based on the identified at least one technical factor or the at least one clinical factor; responsive to the trigger condition being satisfied, automatically causing, on the display device, a display of a graphical window that overlays and prevents interaction with at least a portion of the user interface on the display device, and which presents a control configured to, when activated, reduce the number of workflow steps that are required to identify the type of the infusion and to switch the infusion device from the first mode to a second mode; receiving, after the graphical window is displayed, an indication that the control was activated and that the type of the infusion was identified via the reduced number of workflow steps being completed by a user; and switching the infusion device from the first mode to the second mode based on the type of the infusion and the reduced number of workflow steps being completed by the user.

Clause 20. A non-transitory machine-readable medium comprising instructions thereon that, when executed by one or more processors, cause the one or more processors to perform a method according to Clause 19.

Clause 21. The non-transitory machine-readable medium of Clause 20, wherein the trigger condition comprises an expiration of a timer, further comprising: receiving, while the control is not displayed, an indication of a user authentication to the infusion device or that another infusion on a second channel of the infusion device was initiated in or updated to the second mode; and bypassing the timer to cause the display of the control responsive to the indication.

Clause 22. The non-transitory machine-readable medium of Clause 20 or Clause 21, wherein causing the display of the control comprises: causing a graphical interface to be displayed on a mobile device assigned to a clinician associated with the infusion device, wherein activation of the control and completion of the user workflow is received at the mobile device.

### FURTHER CONSIDERATION

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The predicate words “configured to”, “operable to”, and “programmed to” do not imply any particular tangible or intangible modification of a subject, but, rather, are intended to be used interchangeably. For example, a processor configured to monitor and control an operation, or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such as an “embodiment” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

As used herein a “user interface” (sometimes also referred to as an interactive user interface, a graphical user interface or a UI or a screen) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, .NET™, C, C++, web services, or rich site summary (RSS). In some embodiments, a UI may be included in a stand-alone client (for example, thick client, fat client) configured to communicate (e.g., send or receive data) in accordance with one or more of the aspects described. The communication may be to or from a medical device or server in communication therewith.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, displaying, and the like via a hardware element or causing a hardware element to take one of these actions.

As used herein, the term “message” encompasses a wide variety of formats for communicating (e.g., transmitting or receiving) information. A message may include a machine readable aggregation of information such as an XML document, fixed field message, comma separated message, JSON, a custom protocol, or the like. A message may, in some implementations, include a signal utilized to transmit one or more representations of the information for the message. While recited in the singular, it will be understood that a message may be composed, transmitted, stored, received, etc. in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some implementations, an n-input switch may be included to provide selective functionality where n is the number of inputs used to make the selection.

As user herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

As used herein, “real-time” or “real time” may refer to availability for processing at or near in time to the time the associated item is generated or detected or otherwise available.

In any embodiment, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

What is claimed is:

1. An infusion device configured to:
detect an infusion was initiated by the infusion device in a first mode, wherein when the infusion device is in the first mode a type of the infusion is unknown to the infusion device;
cause, on a display device associated with the infusion device, a display of a user interface comprising real time information pertaining to the infusion, the user interface providing an interactive workflow comprising a number of workflow steps that are required to select the type of the infusion and to switch the infusion device from the first mode to a second mode that is based on the selected type of the infusion;
when the infusion is initiated in the first mode:
generate a trigger condition based on at least one technical factor associated with the infusion device or at least one clinical factor associated with a patient receiving the infusion or a care area or clinician associated with the infusion device;
responsive to the trigger condition being satisfied, automatically cause, on the display device, a display of a graphical window that presents a control configured to, when activated, prompt a selection of the type of the infusion to switch the infusion device from the first mode to a second mode;
receive, after the graphical window is displayed, an indication that the control was activated and that the type of the infusion was selected; and
switch the infusion device from the first mode to the second mode based on the selected type of the infusion.

2. The infusion device of claim 1, wherein the infusion device is further configured to:
identify, when the infusion is initiated in the first mode, the at least one technical factor or the at least one clinical factor;
reduce, when the control is activated, the number of workflow steps that are required to select the type of the infusion to switch the infusion device from the first mode to the second mode,
wherein the infusion device is switched from the first mode to the second mode via the reduced number of workflow steps being completed by a user.

3. The infusion device of claim 1, wherein the at least one clinical factor comprises the infusion device being assigned to a care area associated with high risk patients, the infusion device being further configured to:
receive an indication that the infusion device is assigned to a care area associated with high risk patients; and
initiate a timer responsive to the indication that the infusion device is assigned to the care area associated with high risk patients, wherein the trigger condition comprises an expiration of the timer.

4. The infusion device of claim 1, wherein the at least one clinical factor comprises a shift change within the care area that affects a clinician associated with the infusion device, the infusion device being further configured to:
identify the care area associated with the infusion device;
determine an occurrence of the shift change within the identified care area that affects the clinician associated with the infusion device; and
initiate a timer responsive to the occurrence of the shift change, wherein the trigger condition comprises an expiration of the timer.

5. The infusion device of claim 1, wherein the at least one clinical factor comprises receiving input of an atypical parameter value, the infusion device being further configured to:
receive an operational parameter for administering the infusion;
determining that a value of the received operational parameter is atypical of historical values for the operational parameter; and
initiate a timer responsive to determining that the value of the received operational parameter is atypical of historical values for the operational parameter, wherein the trigger condition comprises an expiration of the timer.

6. The infusion device of claim 1, wherein the at least one clinical factor comprises receiving input of an atypical infusion rate, the infusion device being further configured to:
receive an input of a rate for the infusion;
determine that the rate for the infusion satisfies a threshold rate; and
initiate a timer responsive to determining that the rate for the infusion satisfies the threshold rate, wherein the trigger condition comprises an expiration of the timer.

7. The infusion device of claim 1, wherein the at least one technical factor comprises the infusion device being programmed for an infusion rate that is atypical for an infusion source loaded into the infusion device, the infusion device being further configured to:
receive an indication of a type of the infusion source loaded into the infusion device;
receive an input of the infusion rate;
determine that the infusion rate is atypical for the type of the infusion source loaded into the infusion device; and
initiate a timer responsive to determining that the infusion rate is atypical for the type of the infusion source loaded into the infusion device, wherein the trigger condition comprises an expiration of the timer.

8. The infusion device of claim 7, wherein the infusion device comprises a syringe pump and the infusion source is a syringe, the infusion device further configured to:
determine a size of the syringe loaded into the infusion device,
wherein determining that the infusion rate is atypical for the type of the infusion source comprises determining that the infusion rate is above a threshold rate that is predetermined for the size of the syringe.

9. The infusion device of claim 1, wherein the trigger condition comprises an expiration of a timer, the infusion device being further configured to:
receive an indication of a user authentication to the infusion device; and
bypass the timer to cause the display of the graphical window responsive to the indication of the user authentication.

10. The infusion device of claim 1, wherein the trigger condition comprises an expiration of a timer, and wherein the infusion is initiated on a first channel of the infusion device, the infusion device being further configured to:
receive an indication that another infusion on a second channel of the infusion device was initiated in or updated to the second mode; and
bypass the timer to cause the graphical window to be displayed responsive to the indication that the other infusion was initiated in or updated to the second mode.

11. The infusion device of claim 1, wherein causing the display of the graphical window comprises:
causing the graphical window to be displayed on a mobile device assigned to a clinician associated with the infusion device, wherein activation of the control and completion of the workflow steps is received at the mobile device.

12. A machine-implemented method, comprising:
detecting an infusion was initiated by an infusion device in a first mode, wherein when the infusion device is in the first mode a type of the infusion is unknown to the infusion device;
responsive to the infusion device being initiated in the first mode, generating a trigger condition based on at least one technical factor associated with the infusion device or at least one clinical factor associated with a patient receiving the infusion or a care area or clinician associated with the infusion device;
responsive to the trigger condition being satisfied, automatically causing, on a display device associated with the infusion device, a display of a control configured to, when activated, provide a user workflow to select the type of the infusion and to switch the infusion device from the first mode to a second mode;
receiving, after the control is displayed, an indication that the control was activated and that the type of the infusion was selected via the user workflow being completed by a user; and
switching the infusion device from the first mode to the second mode based on the selected type of the infusion.

13. The machine-implemented method of claim 12, wherein the trigger condition comprises an expiration of a timer, the method further comprising:
responsive to the expiration of the timer, causing the display device to display a prompt to switch the infusion device from the first mode to the second mode together with the control, and restricting use of the infusion device by the user until the prompt is acknowledged by the user.

14. The machine-implemented method of claim 12, wherein the trigger condition comprises an expiration of a timer, and wherein the at least one clinical factor comprises:
the infusion device being assigned to a care area associated with high risk patients,
a shift change within the care area that affects a clinician associated with the infusion device, or
receiving input of an atypical parameter value,
wherein the method further comprises detecting an occurrence of the at least one clinical factor, wherein the timer is initiated responsive to the occurrence of the at least one clinical factor.

15. The machine-implemented method of claim 12, wherein the trigger condition comprises an expiration of a timer, and wherein the at least one technical factor comprises detecting an atypical infusion source being loaded into the infusion device, wherein the method further comprises detecting an infusion source was loaded into the infusion device and that the infusion source is atypical for historical infusion sources associated with the infusion device.

16. The machine-implemented method of claim 12, wherein the trigger condition comprises an expiration of a timer, the method further comprising:
receiving, while the control is not displayed, an indication of a user authentication to the infusion device; and
bypassing the timer to cause the display of the control responsive to the indication of the user authentication.

17. The machine-implemented method of claim 12, wherein the trigger condition comprises an expiration of a timer, and wherein the infusion is initiated on a first channel of the infusion device, the infusion device being further configured to:
receiving, while the control is not displayed, an indication that another infusion on a second channel of the infusion device was initiated in or updated to the second mode; and
bypassing the timer to cause the control to be displayed responsive to the indication that the other infusion was initiated in or updated to the second mode.

18. The machine-implemented method of claim 12, wherein causing the display of the control comprises:
causing a graphical interface to be displayed on a mobile device assigned to a clinician associated with the infusion device, wherein activation of the control and completion of the user workflow is received at the mobile device.

19. A non-transitory machine-readable medium comprising instructions thereon that, when executed by one or more processors, cause the one or more processors to perform a method, comprising:
detecting an infusion was initiated by an infusion device in a first mode, wherein when the infusion device is in the first mode a type of the infusion is unknown to the infusion device;
causing, on a display device associated with the infusion device, a display of a user interface comprising real time information pertaining to the infusion, the user interface providing an interactive workflow comprising a number of workflow steps that are required to identify the type of the infusion and to switch the infusion device from the first mode to a second mode that is based on the identified type of the infusion;
when the infusion is initiated in the first mode:
identifying at least one technical factor associated with the infusion device or at least one clinical factor associated with a patient receiving the infusion or a care area or clinician associated with the infusion device; and
generating a trigger condition based on the identified at least one technical factor or the at least one clinical factor;
responsive to the trigger condition being satisfied, automatically causing, on the display device, a display of a graphical window that overlays and prevents interaction with at least a portion of the user interface on the display device, and which presents a control configured to, when activated, reduce the number of workflow steps that are required to identify the type of the infusion and to switch the infusion device from the first mode to a second mode;
receiving, after the graphical window is displayed, an indication that the control was activated and that the type of the infusion was identified via the reduced number of workflow steps being completed by a user; and
switching the infusion device from the first mode to the second mode based on the type of the infusion and the reduced number of workflow steps being completed by the user.

20. The non-transitory machine-readable medium of claim 19, wherein the trigger condition comprises an expiration of a timer, further comprising:
receiving, while the control is not displayed, an indication of a user authentication to the infusion device or that another infusion on a second channel of the infusion device was initiated in or updated to the second mode; and
bypassing the timer to cause the display of the control responsive to the indication.
